(19) United States
(12) Patent Application Publication
(10) Pub. No.: US 2002/0025561 A1
(43) Pub. Date: Feb. 28, 2002

(54) Vectors for gene-self-assembly

(76) Inventor: Clague Pitman Hodgson, Omaha, NE (US)

Correspondence Address: Clague P. Hodgson, 4701 Innovation Drive, Lincoln, NE, 68521, US

(21) Appl. No.: 09/836,737

(22) Filed: Apr. 17, 2001

Related U.S. Application Data
(60) Provisional application No. 60/197,882, filed on Apr. 17, 2000.

(51) Int. Cl. C12P 19/34; C12N 15/86; G06F 19/00; G01N 33/48; G01N 33/50

(52) U.S. Cl. 435/91.1; 435/320.1; 702/20

(57) ABSTRACT

The present invention provides novel vectors and methods for assembling complex DNA molecules starting with a plurality of input gene sequences. The input gene sequences (which overlap with each other by a defined number of bases) are cloned into a vector at a unique restriction site that is flanked on each side by class IIS restriction endonuclease sites. When the clones are digested with the class IIS restriction enzyme, the inserts are released from the vector with a defined number of bases removed from either the 5′ or 3′ termini, corresponding to the overlap sequences. The overlap sequences, which are unique, non-palindromic sequence strings, permit the fragments to self-assemble. When the fragments are ligated, a seamless, unambiguous linear array fragments is created. The invention can be used for assembling synthetic genes, constructs, vectors and chromosomes.

### CROSS-REFERENCE TO RELATED APPLICATIONS

This application is entitled to the benefit of Provisional Patent Application Ser. No. 60/197,882, filed Mar. 17, 2000.

### STATEMENT REGARDING FEDERALLY SPONSORED RESEARCH OR DEVELOPMENT

The United States Government (The National Institutes of Health, and The National Institute of General Medical Sciences) funded a portion of the work leading to this invention through NIH Grant No. 1R43 GM58361, and therefore it retains certain property rights in the invention.

### BACKGROUND

### FIELD OF THE INVENTION

This invention relates to recombinant DNA technology, biotechnology, and gene therapy, specifically to an improved method and vectors for seamlessly assembling a plurality of DNA fragments in the correct order and orientation.

### BACKGROUND

Recombinant DNA (rDNA) technology (Cohen and Boyer, 1980) utilizes the ability of restriction enzymes and nucleic acid ligating enzymes to create novel nucleic acid molecules, or ‘designer genes’. This technology for recombining genes is the basis for the biotechnology industry, which uses recombinant DNA molecules to produce novel gene products in genetically engineered cells and organisms. To create novel recombinant DNA molecules, two or more input DNA molecules are ligated (joined) together by means of an enzyme such as DNA ligase. Usually, one of the DNA molecules is a so-called vector molecule, such as a bacterial plasmid (virus) DNA molecule. The vector usually must meet several criteria in order to function. It must: 1) be capable of replicating in an organism (such as E. coli); 2) have a selectable gene marker, such as an antibiotic resistance gene; and 3) have a unique cloning site, where (a) foreign gene(s) can be inserted. The process of introducing foreign genes into a vector is called sub-cloning. For example, a novel gene may be introduced into a vector in a first sub-cloning step. After characterizing the resulting clone, a second DNA molecule may be introduced into the vector, for example, a transcriptional promoter gene sequence might be introduced at the 5′ end of the novel gene that was introduced in the first sub-cloning. In subsequent sub-cloning steps, other DNA sequences are sequentially introduced and characterized.

The process of sub-cloning is time consuming and costly, because each sub-cloning step must be performed sequentially, and each clone must be characterized (for example, by restriction enzyme analysis and DNA sequencing) before the next step can be undertaken. One of the problems with the approach is that traditional restriction enzymes (class II enzymes) often digest the ends of the DNA molecules so as to produce either blunt ended molecules, or molecules with short, protruding termini. Protruding termini are usually palindromic, or symmetrical. A DNA molecule with similar protruding palindromic termini can ligate to itself, or to another molecule having the same palindromic or blunt termini. Furthermore, palindromic or blunt termini lack directionality, so they can ligate in either orientation. Any number of molecules with the same terminal extension can ligate to each other in either orientation. Thus, if two molecules with similar ends (blunt or palindromic) are combined in a cloning reaction, the number of permutations and combinations is very large, necessitating undue screening and characterization steps in order to identify the correct products. The major flaw of the traditional cloning process is the use of random probability to create the desired molecules. This has delayed the development of successful gene therapy, where complex vectors must be constructed for use in humans. Herpes simplex virus I (HSV-1, 150 kilobases) and Adenovirus 5 (36 kilobases) are examples of promising vectors for human gene therapy that have yet to be perfected, primarily because of the failure of traditional recombinant DNA technology to permit precise manipulation of these viral genomes.

Ideally, recombinant DNA technology should provide for seamless, unambiguous assembly of many DNA molecules in a single step. This would be possible if palindromes and blunt-ended DNA molecules were eliminated from the cloning process, and if the input DNA molecules (vectors and inserts) instead had unique address tags at their termini, which would allow them to base pair with complementary DNA molecules.

Fortunately, there is a well-known sub-class of class II restriction enzymes (class IIS) that are able to create non-palindromic termini at a designated location (for a review of class IIS enzymes, see Szybalski, 1991). These enzymes cleave the DNA a short distance from the restriction enzyme recognition site, rather than cleaving inside the recognition site. Usually, the class IIS recognition sequence is not a symmetrical sequence, and the enzyme digests the DNA at a precisely defined distance in one direction from the site, leaving a characteristic overhanging end from 1-5 bases in length. Gene amplification (PCR) technology can be performed using primers with class IIS enzyme sites. Because the class IIS sites can be placed anywhere in the primer, it is possible to define a digestion site at a defined distance from the class IIS site, inside the PCR product. Thus, using PCR one can digest a DNA molecule at any site, regardless of whether there is a pre-existing convenient restriction site.

Tomic (Nucleic Acids Res. 18:1656, 1990) reported a method for site-directed mutagenesis using the polymerase chain reaction and class IIS enzymes to join two nucleic acid molecules seamlessly. Lebedenko et al. (Nuc. Acids Res. 19:6757-6771) used class IIS enzymes and PCR for precisely joining 3 nucleic acid molecules for conventioal sub-cloning using BamHI (a regular class II enzyme).

Although a number of investigators successfully used class IIS enzymes for cloning, the construction of complex DNA molecules by this approach was limited to a small number of examples. These are reviewed in: Berlin, Y., DNA Splicing by Directed Ligation, in Genetic Engineering with PCR, R. Horton, [ed.], Horizon Press, Wymondham, England, 1998). In summary, early investigators in the field recognized the ability class IIS enzymes to perform seamless cloning, but were confounded by the permuting effects of palindromes and blunt molecules, as well as by the use of address tags (overhang sequences) more than once.

Ideally, it would be desirable to have a vector with a cloning site flanked by class IIS enzyme sites, permitting the ends of insert molecules to be trimmed, generating overhanging ends that could be programmed with exact address tags. This would be possible if a unique restriction site were placed at the exact site in a plasmid where flanking class IIS enzymes digest.

Mandecki and Bolling developed a method for making synthetic genes by cloning oligonucleotides into a vector with flanking FokI sites (Gene 68:101-107). The authors devised a homologous recombination (gap repair) method for introducing single-stranded ODNs with 15 bp of homology to the cloning site at each end. The authors also pointed out in discussion that a fragment cloned into the SmaI site of their FokI vectors would have four nucleotides cleaved from the 3′-ends when digested with FokI, and thus could be used to construct a series of overlapping fragments Unfortunately, FokI has a 5 bp recognition sequence, causing internal, conflicting sites (FokI sites occur on average every 1,024 bases). In fact, the pUC9 vector backbone (Vierra and Messing, Gene 19:259-268, 1982) used by Mandecki and Bolling to make the FokI vectors has five natural FokI sites, in addition to the two such sites introduced into the cloning site during construction of the vector. Thus, digestion of these vectors with the enzyme FokI would lead to seven fragments, including six from the vector and one to be recovered from the insert site. It would thus be difficult to purify insert fragments from the plethora of fragments generated by FokI digestion of these plasmids. This may explain why the vectors of Mandecki et al. were not used for generating purified fragments larger than about 100 bp. Another problem related to the use of these vectors is that they generate 4 base overhanging ends, which are conducive to the accidental inclusion of palindromic overhanging ends (these must be eliminated, as they self-ligate, and ligate in either orientation).

In order to precisely join a plurality of fragments using class IIS enzymes, it was necessary to eliminate three things: palindromes, blunt ends, and repeated address tags (see Hodgson, PCT/US9803918). Using this approach, one-half dozen fragments can be assembled and introduced directly into mammalian cells, without the need for using a prokaryotic host such as E. coli as a cloning intermediate. Functional selection of the finished product can be substituted for characterization of individual clones after each sub-cloning step. Gene self-assembly technology is described in detail in the Hodgson specification cited Supra, which is incorporated herein as if fully set forth. The ability to construct complex DNA molecules seamlessly (without linkers or adapters) can speed the production of vectors needed, for example, in human gene therapy. Thus, the technology uses class IIS enzymes and PCR as an effective substitute for repeated sub-cloning.

However, gene self-assembly involves first synthesizing the input DNA molecules (as ODNs or as ODN/PCR products), and these processes can cause mutations in one or more of the DNA fragments that make up a gene construct. Ideally, it would be desirable to combine gene self-assembly technology with a process for eliminating mutations before the fragments were joined together. One could then be assured that a correctly assembled construct had the highest probability of success. For example, a number of DNA molecules (comprising a designed gene construct) could be: 1) cloned into a vector with a unique cloning site flanked by class IIS sites not found elsewhere in the vector; 2) sequenced using standard primers and sequencing protocols; 3) digested with a class IIS enzyme (or enzymes), liberating the fragments and the unique address tags (without fragmenting the vector); and 4) ligated to yield the desired construct. Ideally, the vectors should be digested with a rare-cutting enzyme (such as the 7-base recognizing enzyme, Sap1), producing DNA molecules for gene assembly that have protruding ends of a defined length. Ideally, the vector would not contain any internal sites recognized by the chosen class IIS enzyme(s) (other than those flanking the cloning site), or the enzyme used to clone the fragments into the vector. Ideally, the length of the overlapping address tags would be an odd number of bases, eliminating palindromic sequences (palindromes must have an even number in order to conserve symmetry).

### SUMMARY

The invention provides a means for assembling a plurality of DNA fragments seamlessly, in an unambiguous order and orientation, by means of a vector. The vector facilitates the placement of unique, non-palindromic address tags on the ends of the fragments, and allows validation of the fragments prior to assembly to assure that the resulting construct that is free of mutations.

### OBJECTS AND ADVANTAGES

Accordingly, several objects and advantages of the invention are:

(a) to provide vectors for gene self-assembly, which trim a defined number of bases from the ends of nucleic acid strands, providing complementary ends that fit together in a defined order and orientation;

(b) to provide a method for using the vector to prepare constructs having a unique and unambiguous structure

(c) to provide a method for efficiently assembling a plurality of DNA sequences into a construct, vector, or chromosome;

(d) to provide a method for seamlessly joining a plurality of DNA sequences at exact nucleotide positions;

(e) to provide a method for combinatorial assembly of a plurality of DNA fragments;

(f) to provide a method for color selection of clones containing inserts; and

(g) to provide a method for determining that DNA fragments are free of mutations prior to building the construct.

Further objects and advantages are to decrease the cost and time needed to complete complex cloning jobs, and to facilitate production of vectors that are needed for gene therapy, transgenesis and biotechnology. Still further objects and advantages will become apparent from a consideration of the ensuing description and drawings.

### DESCRIPTION-DRAWING FIGURES

### BRIEF DESCRIPTION OF THE DRAWING FIGURES

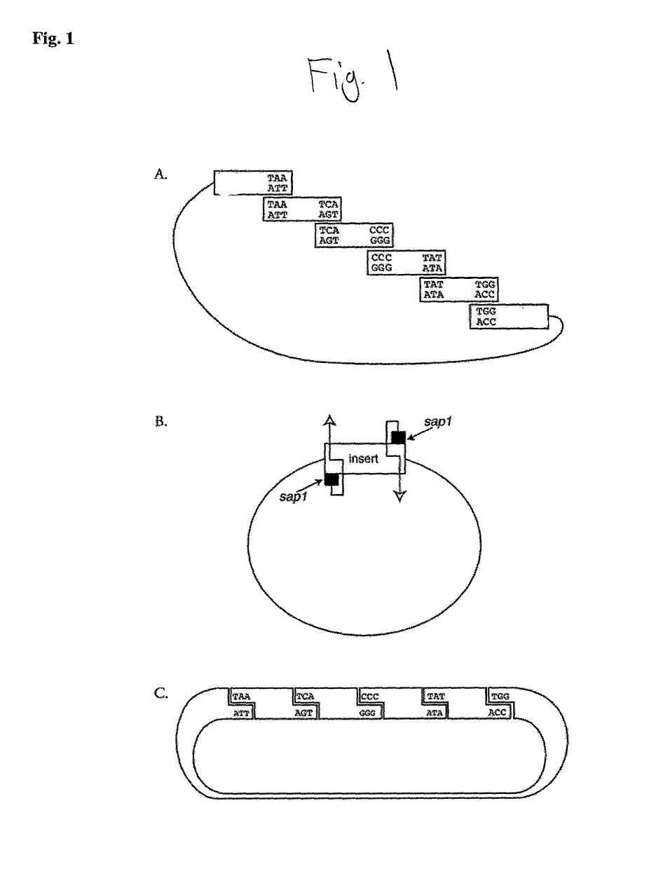

FIGS. 1A to 1C show how a series of overlapping fragments are cloned into a vector and trimmed with a restriction enzyme so as to fit together in a definite order and orientation.

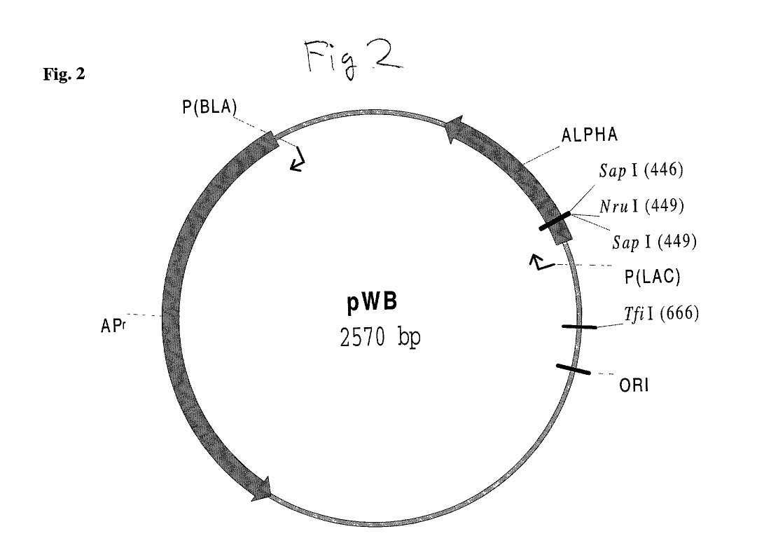

FIG. 2 shows the vector pWB (SEQ ID NO:1).

### DETAILED DESCRIPTION OF THE DRAWING FIGURES

FIG. 1. Schematic of the vector method of gene self-assembly. (A) A series of blunt, phosphorylated DNA fragments is prepared, the members of which overlap by a defined number of bases (e.g., three bases). (B) The fragments are individually cloned into a unique cloning site in the vector (vector is indicated by the curved line). Each of the cloned inserts are digested with the appropriate class IIS restriction enzyme (SapI in the example), releasing the DNA fragments that now have been modified by removal of the defined number of bases (constituting the overlap sequences) from both ends of the fragments (either the 5′- or 3′ termini can be shortened). (C) The cloned insert DNA fragments, digested with the class IIS enzyme, are isolated from the vector sequences, and are annealed and ligated together, creating a gene, construct, vector, or chromosome (either linear or circular).

FIG. 2. A circular map of the preferred vector pWB. (SEQ ID NO:1) p(BLA)=The promoter of the β-lactamase gene (APr). Ori=The origin of replication of the plasmid DNA. ALPHA=the alpha subunit of the β-galactosidase gene. SapI=SapI restriction endonuclease recognition sites. NruI=NruI restriction endonuclease recognition site, which is the unique, blunt cloning site for this vector. P(LAC)=The .00. NruI site, located between the two SapI sites in the cloning site sequence:

GCTCTTCGCGAAGAGC (SEQ ID NO:2). This sequence was extended by four bases on each end (24 base pairs, total), to preserve the open reading frame of the ALPHA gene, creating the overall ODN sequence, GGAAGCTCTTCGCGAAGAGCTTCC (SEQ ID NO:3). Important features of this vector are the lack of NruI or SapI cleavage sites outside of the cloning/trimming site.

### DETAILED DESCRIPTION OF THE INVENTION

The present invention (FIG. 1) provides novel methodologies and vectors for creating nested sets of gene fragments that can be assembled seamlessly and unambiguously.

A preferred embodiment of the invention is a process for assembling genes, comprising, first, constructing a set of DNA molecules that are double-stranded, blunt, and that have unique, non-palindromic overlapping sequences shared with adjacent fragments at 5′- and 3′-ends (FIG. 1A). Second, the fragments are individually cloned into a unique site within the vector (such as the unique site of pWB, FIG. 1B, SEQ. ID. NO: 1), that is flanked by class IIS enzyme sites that do not occur elsewhere in the vector, and that are positioned to allow removal of the insert sequences by digestion with the class IIS enzyme, providing single-stranded termini on the insert sequences that fit together like jigsaw puzzle pieces. Third, the individual cloned inserts are preferably sequenced, to determine whether or not they are free from mutations. Fourth, a single characterized clone of each insert fragment is then digested with the class IIS enzyme defined by the vector (such as the SapI sites of pWB), releasing the DNA fragment from the vector. Fifth, the released insert fragments are isolated and purified from the vector fragments (for example, by isolation from an agarose gel, or by ion-pair reverse-phase high performance liquid chromatography [i.e., the WAVE machine, Transgenomic, Inc., Omaha, Nebr.]). Sixth, the fragments are combined, preferably in a 1:1 molar ratio, and are annealed and ligated together, for example using bacteriophage T4 DNA ligase and ATP (available from New England Biolabs, Beverly, Mass.), thus creating the desired construct.

The completed gene construct can then be used for a variety of purposes. For example, it can be inserted into a vector. Conversely, one or more of the insert pieces may comprise the vector, and the construct is thus a complete recombinant DNA molecule/vector. The construct can be made so as to be either a linear or a circular molecule (in the latter case, the first insert fragment also joins with the last [FIG. 1C], making it circular). For example, up to 32 fragments can be assembled in a unique construct using the vector pWB. There is no upper size limit as to the size of the fragments. The input fragments can be any blunt DNA molecules: PCR products, blunt restriction fragments, synthetic double-stranded ODNs, or the like.

It is theoretically possible to make a chromosome or mini-chromosome, starting with a number of fragments. The essential parts of a chromosome are: telomeres (terminal repeat sequences), centromere, and origins of DNA replication. One or more, or a plurality of genes may also be included. Mini-chromosomes used for gene engineering may be circular or linear. Circular constructs need not contain telomeres, which are found at the ends of linear chromosomes. The assemblies may be made up of sub-assemblies, and the sub-assemblies may themselves be assembled and cloned inside a vector such as pWB. The sub-assemblies may then be given unique address tags by the methods of the instant invention, and a super-assembly of these sub-assemblies may be made. Thus, the construct can be grown in steps to any desired size or complexity. If the sub-assemblies are larger than approximately 30 kb, it may be desirable to place the cloning/class IIS digestion sites of the invention into a cosmid vector backbone (after removing competing class IIS sites, if necessary) for the initial cloning step.

If the construct is larger than about 40-50 kilobase pairs, it may be desirable to use a bacterial artificial chromosome (BAC) containing the class IIS sequences and cloning site such as SEQ ID NO:2. The purpose of a BAC vector, rather than a plasmid replicon vector is that a BAC vector can hold several hundred kilobases of DNA. Thus, one set of genes may be assembled, and the sub-assembly then becomes an input fragment for the next level of assembly, until the chromosome is built. For example, several sub-assemblies cloned into, for example, a BAC containing the SapI/NruI sequence (SEQ ID NO:2), and they can be removed from the BAC by digestion, and joined together, creating a chromosome or mini-chromosome. A mini-chromosome may be up to one or more megabases, while a chromosome may be up to 100 megabases or more in size. Once the chromosome is assembled, the ligation mix can be injected directly into an egg or other cell of an animal, bacterium or plant, creating a transgenic cell, egg, or organism.

One or more endogenous chromosomes of the recipient cell or egg can be irradiated or treated with laser irradiation, for example, in order to destroy the endogenous chromosome and effect a chromosome replacement with the assembled chromosome in place of the endogenous chromosome. If replacement is effective, the replacement should restore viability and therefore be auto-selecting.

In order to prevent shearing of a mini-chromosome or chromosome joined by ligation in vitro, it may be desirable to add a DNA condensing agent (such as Lipofectamine™, Life Technologies, Inc.,) or an equivalent reagent, such as a polycationic DNA condensing agent, prior to handling the assembled DNA. This will have the effect of condensing the DNA into a small particle, preventing shearing and permitting transfer to living cells/organisms. Ideally, the chromosome should contain at least one gene that is a selectable marker (such as the neomycin phosphotransferase gene [Clontech Labs, Mountain View, Calif.]), and/or a gene that is a reporter gene (such as the green fluorescent protein [GFP]gene [Clontech Labs]), so that the cells expressing and maintaining the gene may be easily detected and/or purified.

In another preferred embodiment, one or more of the insert fragments comprises a fragment set or pool, rather than an individual fragment, permitting a number of permutations of fragments to be made, such as a combinatorial library. This, and other embodiments of gene self-assembly are described in Hodgson, PCT/US9803918, above, and are incorporated herein as if fully set forth.

In another preferred embodiment, the insert DNA fragments are derived by PCR, using primer sequences to create overlapping sequence between the sequences to be joined. The PCR enzyme Pfu DNA polymerase (Stratagene, La Jolla, Calif.) is a preferred enzyme, because it creates DNA molecules that terminate exactly with the primer termini, and which are not extended by any base pairs beyond the primer-template sequence. Extension of even one base is a problem associated with enzymes such as Taq DNA polymerase, because such extension destroys the perfect spacing that is necessary with unique address tags. Taq DNA polymerase is also more error-prone than the high-fidelity, preferred Pfu DNA polymerase. Taq enzyme should therefore not be used with vectors such as those of the instant invention, because it destroys the precise spacing required by the method.

However, Taq DNA polymerases (especially Taq DNA polymerase mixes containing a proof-reading DNA polymerase enzyme, which are more efficient) can be used in practicing the invention if a T-A cloning site (single 3′-T overhangs on both 3′-strands) is located between the class IIS sites, permitting T-A cloning to be used (Invitrogen Corporation, Carlsbad, Calif.). Taq-amplified PCR products have a single 3′-A residue that will join with such vectors, permitting Taq products to be cloned effectively in a vector of the instant invention. The vectors can be provided with topoisomerase I enzyme, permitting the ends of both vector and insert to be efficiently joined, with minimal effort.

If PCR is used to make the input DNA fragments, the PCR products are preferably purified from an agarose (electrophoresis) gel slice containing the fragment (using, for example, a Qiagen gel DNA purification kit, Qiagen, Inc., Valencia, Calif.) to remove impurities. If PCR is used, SYBR green I dye (Molecular Probes, Eugene, Oreg.) is preferably used for visualizing the DNA in agarose gels, in place of the traditional ethidium bromide stain. This method is preferred because SYBR green I fluorescence provides greater sensitivity, is non-mutagenic, and can be visualized without ultraviolet light (in order to minimize damage to the DNA fragments during isolation). Preferably, the fragments are stored in tris-EDTA buffer (TE buffer, 10 mM Tris base, 1 mM EDTA, pH 8.0), rather than in water or a more dilute buffer (TE storage minimizes degradation of the ends of the DNA molecules). Standard buffers and reagents for recombinant DNA work can be found in Ausubel (Current Protocols in Molecular Biology, 2000).

In yet another preferred embodiment, the insert DNA fragments are created from synthetic oligodeoxynucleotides (ODNs), by means of any methods for using either annealed, double-stranded ODNs, or DNA molecules made by polymerizing annealed ODN sequences to fill gaps. Synthetic ODNs can be ordered from a commercial supplier (e.g., MWG, Inc., Charlotte, N.C.), and should be purified by a fastidious process, such as by MWG's standard, high-purity salt-free (HPSF) process, prior to cloning. This approach allows the construction of large, synthetic DNA molecules. Fragments of 100-300 bp are first made using double-stranded ODNs, or by splice overlap extension methods (reviewed in Berlin, Supra). The ODNs are phosphorylated using polynucleotide kinase (New England Biolabs) and are cloned into a vector such as the preferred pWB. DNA sequencing of the insert fragments is used to eliminate mutations. Larger constructs are then assembled using the methods and vectors described in the instant invention.

In yet another preferred embodiment, the cloning site consists of an NruI blunt cloning site, flanked with SapI sites overlapping the NruI site, as in the sequence of SEQ ID NO:2. To wit, that sequence GCTCTTCGCGAAGAGC consists of a SapI recognition site (GCTCTTC), an overlapping NruI site (TCGCGA), and a second overlapping SapI site on the opposite strand (GAAGAGC). The two SapI sites are poised to digest the DNA from both sides. When an insert is ligated into the blunt NruI site, it is spaced exactly one base pair away from the SapI recognition sites. This is exactly the length of the ‘arm’ between the recognition site and the digest site. The SapI digestion site is: GCTCTTC N^{ˆ} NNNA_{ˆ} , where SapI digests a 5′-overhang of 3 bp. This exact sequence can also be modified by adding any number of nucleotides between the NruI site and a second NruI site, followed by a second SapI site that is inverted with respect to the first. Thus, an important aspect of the invention is the half-site GCTCTTCGCGA (SEQ ID NO:4), which is a combination of a SapI site with an overlapping NruI site. This combination allows trimming of three bases from one end of a nucleic acid molecule cloned in the NruI site. Removal of an insert placed between two, oppositely-facing half-sites then recreates the same vector as merely cutting pWB with NruI. However, it is not necessary to use half-sites, as this creates an additional step to remove the intervening nucleotides before cloning. Thus, the sequence of SEQ ID NO:2 is a preferred sequence for this vector.

An important aspect of the preferred vector pWB is the fact that the cloning site (NruI, a blunt-cutter) is located within and adjacent to and overlapping with the SapI sites. Two SapI sites are aligned in opposite directions, so that they digest toward each other. The NruI site is located between the SapI sites and overlapping them, so that both SapI enzyme sites lead to digestion at the center of the NruI site, and 3 bases beyond it. When a DNA fragment is cloned within the NruI site, the SapI sites are positioned so as to remove three bases from each terminus, on the 3′-strand only. This causes the fragments to fit uniquely together, like the pieces of a jigsaw puzzle. It is not necessary to determine the orientation of cloning fragments into pWB, as the cloning site is symmetrical.

### DESCRIPTION-EXAMPLES

### Example 1: Constructing a Universal Vector (pWB) for Gene Self-assembly

The vector pUC18 (Yanisch-Perron, Gene 33:103-119, 1985; American Type Culture Collection, Rockville, Md., Cat. No. ATCC 37253) was digested with the restriction enzyme TfiI (New England Biolabs), removing a non-essential region of 140 bp containing an undesired Sap1 site. To do so, 35 μl (6.5 μg) was digested with 15 U of TfiI in the buffer supplied by the manufacturer (buffer 3), for 1 h 50 min at the recommended temperature (65° C.) in a 43 μl volume. The reaction was then run on a 1% agarose gel, and the digested fragment was removed from the gel with a scalpel blade and was purified using a QIAquick gel extraction kit (Qiagen, Inc., Valencia, Calif., Cat. No. 28704). A ligation reaction was performed by adding 58 ng (1 μl) of DNA, 3 μl of distilled water, 0.5 μl of a ten times concentrated ligase buffer (with ATP) and 0.5 μl of DNA ligase enzyme (10,000 U, New England Bioloabs). The reaction was allowed to proceed for 110 min at 16° C. Super-competent DH5α E. coli cells (Stratagene, Inc., LaJolla, Calif.) were transformed according to the manufacturer's instructions (50 μl of cells, 2.5 μl of the ligation reaction containing pUC18 digested with TfiI and relegated, as described above). The cells were grown according to the manufacturer's instructions on agar plates, and a colony was isolated, grown, and tested for digestion with Sap1. Lack of digestion, compared to parental pUC18 indicated that the Sap1 site had been eliminated. DNA from this clone (pUC18Sap1-) was digested with restriction enzyme Sma1, linearizing the vector at a prominent cloning site within the pUC18 multiple cloning site. The blunt-ended, linearized, Sma1-digested vector was then treated with calf intestinal phosphatase (CIP, Roche Molecular Biochemicals, Indianapolis, Ind.) to remove 5′-terminal phosphates and to prevent re-ligation of the plasmid. The treated vector was purified using a QLAquick column (Qiagen). A palindromic, 24 base oligonucleotide was ordered from Sigma-Genosys: (SEQ ID NO:3, bases 437-460). 1 μg of this oligonucleotide was phosphorylated with polynucleotide kinase according to the manufacturer's instructions (Boehringer-Mannheim), and was annealed to itself for 15 min at 37° C. 20 ng of the phosphorylated, annealed ODN was added to 1 μg of SmaI digested pUC18Sap1-vector, and ligated with 2,000 U of T4 DNA ligase according to the manufacturer's instructions (New England Biolabs) for 1 h at 16° C. The vector was transformed into E. coli SCS110 cells (Stratagene). A clone was identified that was digestible with Sap1 enzyme, which had two copies of the ODN inserted. This clone was digested with restriction enzyme Nru1 to remove one equivalent insert, and was ligated back together and re-transformed into E. coli SCS110 cells (Dam-, Dcm- [prevents methylation of certain restriction sites], EndA-[for high quality plasmid mini-prep DNA]), producing the desired construct, as identified by sequencing. This vector is pWB, SEQ ID NO:1.

### Example 2: Preparing pWB for Cloning Inserts

100 μg of pWB DNA was digested with Nru1 enzyme in a 200 μl volume for 2 h at 37° C., and was heat-inactivated at 65° C. for 20min. The DNA was then de-phosphorylated with CIP enzyme, as described above. The digested, de-phosphorylated DNA was electrophoresed through a 1% agarose gel to separate digested from residual undigested plasmid. The band representing the Nru1 digested, dephosphorylated plasmid was excised from the gel with a scalpel, and was removed from the gel using a QIAquick gel extraction kit (Qiagen). To insure complete removal of the residual Nru1 enzyme (which can inhibit ligation), the gel slice was dissolved at 60° C. After QIAquick DNA purification, the purified pWB DNA was used for cloning.

### Example 3: Generic Method for Cloning dsDNA Sequences in pWB (Fragments Prepared by any Method)

50 ng of Nru1 digested, de-phosphorylated, gel-purified pWB is ligated to a 5′-phosphorylated insert fragment (if PCR fragments or double-stranded ODNs are used, they must be phosphorylated, for example using the enzyme polynucleotide kinase [NEN]). Preferrably, a 1:1 molar ratio of each fragment is ligated at 16° C. for 1 h with 2,000 U of T4 ligase in ATP-containing buffer (described above). 1-50 ng of the ligation mix is transformed into an E. coli strain such as DH5α. Competent cells can be purchased from a supplier (Stratagene). After transformation, the cells are plated using an appropriate antibiotic (pWB=100 μg/ml ampicillin), using Luria-Bertani media (Miller, Fisher Scientific catalog No. BP1426-500) and agar plates with 15 g of Bacto agar per liter. To obtain blue-white selection, IPTG (inducer) and X-gal (stain) is added to plates when the competent cells are plated (follow manufacturer's instructions, Stratagene).

### Example 4: Choosing Unique Overlaps

Use of a codon table (such as the genetic code table used for translating mRNA sequences [Ausubel, 2,000]) is a convenient method for keeping track of which 3 base overlaps (and their complements) have been used in cloning fragments into pWB. For other enzymes, a table of all possible non-palindromic terminal extensions of the appropriate length can be kept. The purpose of this bookkeeping is so that no pair of overlap sequences is used more than once. In the case of enzymes having 2 or 4 base overlaps (or other even numbers of bases overlapping), it is essential to use a table lacking palindromic sequences (those that can self-ligate, or ligate in either orientation). Failure to keep track of which overlaps have been used (or of palindromes) will likely lead to failure. It is also possible to design a simple computer program that does the same thing, using a computer database or spreadsheet (such as an EXCEL spreadsheet, Microsoft Corp., Everett, Wash.) to keep track of which overlap sequences have been used. In the absence of a computer program, it is very helpful to keep both a list and a paper model, similar to a child's cut-outs, wherein the fragments are shown as in FIG. 1C, with the overlap sequences juxtaposed so that no errors will be made in designing input fragments. A simple list will also suffice to keep track of overlap sequences.

Using the 3 base overlaps defined by SapI, it is possible to join 32 fragments together, using the 64 possible 3-base ‘codons’ in pairs at each junction. Using an enzyme such as Hga1 (five base overlaps, 1024 possible combinations), it is theoretically possible to join 512 fragments. This advantage of HgaI is balanced by the shorter recognition sequence (5 bases, vs. 7 bases for Sap1). In order for an enzyme such as HgaI or FokI to be used with effectiveness, all homologous sites within the vector backbone (except the sites flanking the cloning site) will have to be eliminated. This can be done by deletion (if in a non-essential region, as in the example of pWB), or it can be done by changing codons that contain sites to homologous codons that do not have the sequence of the restriction enzyme. This can be done by PCR, using the method of Tomic (Supra) to change codons.

The artisan is cautioned, when using overlap sequences of any length, to use those overlap sequences that have a higher GC content. The reason is that these have a higher melting temperature (Tₘ), and can thus be annealed and ligated at a higher temperature, favoring more specific interaction and greater enzyme activity. For example, a simple formula suggests that AT base pairs have a Tₘ of approximately 2° C. for each base pair, while GC base pairs have a Tₘ of approximately 3° C. for each base pair. Thus, it is desirable to ligate 3-base AT-only extensions at 6° C., while GC-only extensions could be ligated at approximately 12° C., near to or below the Tₘ.

### Example 5: Blue-white Selection

In the exemplary vector, pWB, a 24 base oligonucleotide (GGAAGCTCTTCGCGAAGAGCTTCC, SEQ ID NO:3) is inserted in-frame in the open reading frame of the E. coli β-galactosidase gene (the oligonucleotide sequence used in pWB corresponds to bases 437-460 in FIG. 3). Thus, the oligo used to make this vector does not interrupt the open reading frame, leaving the β-gal gene intact by inserting 8 codons of new genetic information into the open reading frame. Thus, clones with no insert are blue when X-gal and IPTG are used. However, when inserts are inserted into the vector, the reading frame of the β-gal gene is often interrupted by stop codons, or the biological activity is interrupted by long insertions, resulting in inactivation of the gene. This produces white colonies that can be detected visually. White colonies contain inserts. Thus, white colonies are selected for screening. It is possible that rare inserts are completely in the open reading frame, and will have residual β-gal activity, in which case it will be necessary to screen several clones for inserts. This is made easy by the fact that the dephophporylated vector produces mostly clones with inserts.

### Example 6: Screening for Correct Inserts

Mini-preps of plasmid DNAs are made by growing overnight cultures of white colonies picked from agar plates (Qiagen, mini-prep kit). Several of each fragment cloning experiment are sequenced on an automated sequencer (Applied Biosystems, Palo Alto, Calif.), using standard (forward and reverse) primers. In the case of the preferred pWB, the primers are standard catalog items available from New England Biolabs: forward primer Cat. No. 1212; reverse primer Cat. No. 1233. Generally, any primer sequences can be custom ordered (MWG, Inc.) to suit the vector. A general rule for selecting primers is: 50% GC content; 16-22 bases, no runs of three or more of the same base, primer ends on G or C. If the insert size is greater than about 1 kilobase pairs, internal primers may be necessary to sequence across the entire insert. Sequencing the entire insert is recommended to make sure there are no mutations. Even if this cannot be done, it is desirable to sequence the ends to make sure there is not mutation at the terminus that would alter the overlap sequence.

### Example 7: Harvesting Insert Sequences

Once a collection of clones is obtained wherein at least one representative clone of each fragment has no mutations and has the correct termini, it is time to harvest the inserts. This can be done by any method, but is usually done by digesting the clones with Sap 1, and by running the digested DNA on an agarose gel, such as a 1% agarose gel. The inserts will be visible at the respective position (the vector is 2.6 kb), and are harvested with a scalpel blade. The inserts are collected separately. Gel plugs containing the bands (stained with SYBR green I and visualized with the Dark Reader (Clare Chemical, Denver, Colo.) are removed from the gel are extracted using QIAquick gel extraction kits (Qiagen). If the vector migrates at the same position as the insert, an additional enzyme digest may be used to eliminate the conflicting plasmid band. Internal Sap1 sites in inserts are relatively rare. However, they are occasionally encountered. Usually they present no problem, and they are accounted for in the reservation table to prevent conflicts during design and construction. One way to correct them, if necessary, is to have the sequences begin and end with the natural Sap1 sites, so that the vector cut sites correspond with those of the natural sites. Yet another way is to use the PCR cloning process to erase Sap1 sites found in the inserts. The primer in the synthesis step is used to change one or more bases that make up the Sap1 site. Usually none of these steps are necessary, however. One advantage of the preferred methods and vectors of the instant invention is that Sap1 sites provide an optimal combination of rare cutting (approximately once every 16,000 bp), and 3 bp overlaps (eliminates palindromes), although the actual occurrence of SapI sites in eukaryotic DNA appears to be somewhat higher than expected, possibly due to base composition.

### Example 8: Generic Method for Ligating the Inserts

Once the inserts are harvested, a small disposable sample of each is examined by ultraviolet spectrophotometry (OD260/280), or by fluorescence, to determine the DNA concentration. Approximately equi-molar amounts of inserts are combined. The concentration of DNA should be >20 μg/ml, or more concentrated if fragment size is large or the concentration of ends is low. A preferred ligase enzyme preparation is T4 ligase manufactured by New England Biolabs, Cat. No. 202S or 202CS. Generally, a good way to ligate fragments is to use a PCR machine set for 6-16° C. (depending upon Tm of the overlap sequences) and a set time (overnight is permissible, although 1-2 h is usually sufficient for 3 bp ends to ligate). A preferred method is to set the thermal cycler to start the ligation at 4° C., and step the temperature up by 2° C. for each hour of incubation, up to 16° C., followed by a 4° C. hold. A sample of before and after ligation DNA is saved for gel analysis.

### Notes

The full-length (ligated) DNA band can also be isolated from an agarose gel after the material is ligated, or PCR can be used to amplify the entire assembly from the ligation directly, if too small an amount of full-length product is observed or suspected. A cautionary note is that PCR will introduce mutations. A high fidelity PCR enzyme (preferably Pfu DNA polymerase or cloned Pfu DNA polymerase, and more preferably PfuTurbo DNA polymerase, Cat. No. #600250, Stratagene) should always be used with standard vectors, such as PWB (however, Taq DNA polymerase can be used if the unique cloning site is a T-A cloning site). The Pfu enzymes are said by the manufacturer to have an accuracy rate of approximately 7-8×10⁵ bases/error (bases inserted before an error is made; Stratagene, Inc.).

If the gene assemblage is a vector, it can simply be transformed into the appropriate host cells after ligation. It is not necessary to use E. coli as an intermediate host. Any methods of molecular biology can be invoked to clone or rescue the assembly, including re-cloning of sub-assemblies into the vector pWB. See Ausubel, Supra, for standard instructions relating to molecular biology procedures.

### CONSLUSIONS, RAMIFICATIONS, AND SCOPE OF INVENTION

Thus, the reader will see that the vectors and methods of the invention provide a precise, efficient, and unambiguous way to construct large DNA molecules from a plurality of smaller ones.

It will be readily understood by those of average skill in the art that the foregoing description has been for purposes of illustration only, and that a variety of embodiments can be envisioned without departing from the scope of the invention. For example, viruses can be assembled by the invention, as can chromosomes, synthetic genes, combinatorial libraries, and libraries of exons, introns, antibodies, and promoter elements. Therefore, it is intended that the invention not be limited except by the claims.

I claim:

1. A method for assembling gene constructs from a plurality of DNA fragments, comprising:
(a) preparing a series of overlapping DNA molecules, said DNA molecules having a defined length of overlap, said overlap comprising unique, non-palindromic DNA sequences;
(b) cloning the DNA molecules into a vector, said vector comprising a cloning site that is flanked on both sides by class IIS restriction endonuclease recognition sites, said sites positioned to allow removal by digestion with the class IIS enzyme or enzymes of a defined number of bases from one strand on both ends of the fragment;
(c) validating the insert fragments;
(d) digesting the clones with the appropriate class IIS restriction enzyme or enzymes, releasing the insert DNA fragments, now modified by the removal of the defined number of bases from one strand at each terminus;
(e) purifying the insert fragments away from the vector fragments;
(f) annealing and ligating the insert fragments together; and
(g) characterizing the resulting DNA construct,
Whereby a DNA construct, vector, gene or chromosome with the desired order and orientation of fragments is created.

2. The method of claim 1, wherein the vector is pWB (SEQ ID NO:1).

3. The method of claim 1, wherein the class IIS enzyme site of choice is SapI.

4. The method of claim 1, wherein the vector provides blue-white colony selection to determine whether inserts are present.

5. The method of claim 1, wherein the insert DNA fragments are joined in order by determining non-palindromic overlap sequences from a table, list, chart, or computer database.

6. The method of claim 1, wherein the insert sequences are characterized by means of sequencing or mapping.

7. The method of claim 1, wherein a computer algorithm is used to determine either the choice of primers, sequences, address labels, or the detection of conflicting internal sites within the construct.

8. The composition of matter comprising the plasmid pWB. (SEQ ID No: 1)

9. The composition of matter comprising a vector, said vector comprising a plasmid or bacterial origin of replication, a selectable gene, and a DNA cloning site, said cloning site comprising class IIS restriction endonuclease recognition sites that are not found elsewhere in the vector, flanking at least one unique cloning site,
whereby digesting the vector containing a DNA insert cloned at the said cloning site with the eponymous class IIS restriction endonuclease results in release of the insert from the vector, with removal of a defined number of bases from one strand at each end of the DNA insert sequence.

10. The composition of claim 9, wherein the insertion of a DNA insert causes a change in phenotype that allows identification of clones containing inserts.

11. The composition of claim 9, comprising the sequence: 5′-GCTCTTCGCGAAGAGC-3′ (SEQ. ID. NO: 2).

12. The composition of claim 9, comprising a bacterial origin of replication.

13. The composition of claim 9, wherein said composition is used for the construction of a gene, vector, construct, combinatorial library, or chromosome.

14. The composition of claim 9, comprising two half sites, each half-site comprising the sequence GCTCTTCGCGA (SEQ ID NO:4), wherein the two half sites are located on opposite strands and are separated by one or more nucleotides, wherein the vector is used for trimming one or more bases from the ends of insert fragments.


SEQUENCE LISTING (4 sequences)

SEQ ID NO: 1 - DNA, 2570 bp, Plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagaggatccccggaagctcttcgcgaagagcttccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 16 bp, Plasmid
gctcttcgcgaagagc

SEQ ID NO: 3 - DNA, 24 bp, Plasmid
ggaagctcttcgcgaagagcttcc

SEQ ID NO: 4 - DNA, 11 bp, Plasmid
gctcttcgcga